(19) United States
(12) Plant Patent Application Publication
(10) Pub. No.: US 2017/0042078 P1
(43) Pub. Date: Feb. 9, 2017

(54) Ornithogalum plant named 'MFPRINSTAR'

(50) ;

(71) Applicant: Michael Frank, Moshav Givat Chen (IL)

(72) Inventor: Michael Frank, Moshav Givat Chen (IL)

(21) Appl. No.: 14/756,135

(22) Filed: Aug. 4, 2015

(51) Int. Cl. A01H 5/00

(52) U.S. Cl. PLT/462

(57) ABSTRACT

A new and distinct Ornithogalum cultivar named ‘MFPRINSTAR’ is disclosed, characterized by long inflorescence of 30 cm, height of 60 cm, a high yield of 2-3 flowers per bulb, and light orange flower petals. The new variety is an Ornithogalum, normally produced as an ornamental plant or cut flower.

### LATIN NAME OF THE GENUS AND SPECIES

Ornithogalum dubium

### VARIETY DENOMINATION

‘MFPRINSTAR’

### BACKGROUND OF THE INVENTION

The new Ornithogalum cultivar is a product of a planned breeding program conducted by the inventor, Michael Frank, in Moshav Givat Chen, Israel. The objective of the breeding program was to produce new Ornithogalum varieties for ornamental commercial applications. The cross resulting in this new variety was made during March of 2012.

The seed parent is the, unpatented, commercial variety referred to as Ornithogalum ‘MFGOLDSTAR’. The pollen parent is the unpatented, commercial variety referred to as Ornithogalum ‘MFSSTAR’. The new variety was discovered in March of 2013 by the inventor in a group of seedlings resulting from the 2012 crossing, in a research greenhouse in Moshav Givat Chen, Israel.

Asexual reproduction of the new cultivar was performed by tissue culture. This was first performed at a research laboratory in Moshav Givat Chen, Israel in March of 2013 and has shown that the unique features of this cultivar are stable and reproduced true to type in multiple successive generations.

### SUMMARY OF THE INVENTION

The cultivar ‘MFPRINSTAR’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘MFPRINSTAR’ These characteristics in combination distinguish ‘MFPRINSTAR’ as a new and distinct Ornithogalum cultivar:

1. Light orange petals
2. Long inflorescence (30 cm)
3. Average height—60 cm
4. High yield: 2-3 flowers per bulb
5. 150 days to flowering

Plants of the new cultivar ‘MFPRINSTAR’ are similar to plants of the seed parent, Ornithogalum ‘MFGOLDSTAR’ in most horticultural characteristics, however, plants of the new cultivar ‘MFPRINSTAR’ differ in the following;

1. Light orange flowers, the seed parent has a dark yellow flower.
2. 30 cm long inflorescence, the seed parent has 10 cm long inflorescence.
3. Height is 60 cm, the seed parent is 50 cm.
4. Produces 2-3 flowers per bulb, while the seed parent produces 1-2 flowers per bulb.

Plants of the new cultivar ‘MFPRINSTAR’ are similar to plants of the pollen parent, Ornithogalum ‘MFSSTAR’ in most horticultural characteristics, however, plants of the new cultivar ‘MFPRINSTAR’ differ in the following;

1. Produces 2-3 flowers per bulb, the pollen parent produces 1-2 flowers per bulb
2. Has a 30 cm long inflorescence, the pollen parent has 20 cm long inflorescence.
3. Flowers color is light orange, the pollen parent has orange flowers.

### COMMERCIAL COMPARISON

Plants of the new cultivar ‘MFPRINSTAR’ are comparable to the unpatented commercial variety Ornithogalum ‘MFWHITSTAR’. The two Ornithogalum varieties are similar in most horticultural characteristics; however, the new variety ‘MFPRINSTAR’ differs in the following:

1. The new variety has light orange flowers, while the comparator has white flowers.
2. The new variety height is 60 cm, while the comparator is 55 cm in height.
3. The new variety has smooth stem, while the comparator has a textured stem.
4. The new variety is dubium compatible, while the comparator is dubium incompatible.

Plants of the new cultivar ‘MFPRINSTAR’ can also be comparable to the unpatented commercial variety Ornithogalum ‘MFSSTAR’. The two Ornithogalum varieties are similar in most horticultural characteristics; however, the new variety ‘MFPRINSTAR’ differs in the following:

1. The new variety has light orange flowers, while the comparator has orange flowers.
2. The new variety produces 2-3 stems per bulb, while the comparator produces 1-2 stems per bulb.
3. The new variety flowers after 150 days, while the comparator flowers after 120 days.
4. The new variety is relatively resistant to Erwinia , while the comparator highly sensitive to Erwinia

### BRIEF DESCRIPTION OF THE PHOTOGRAPH

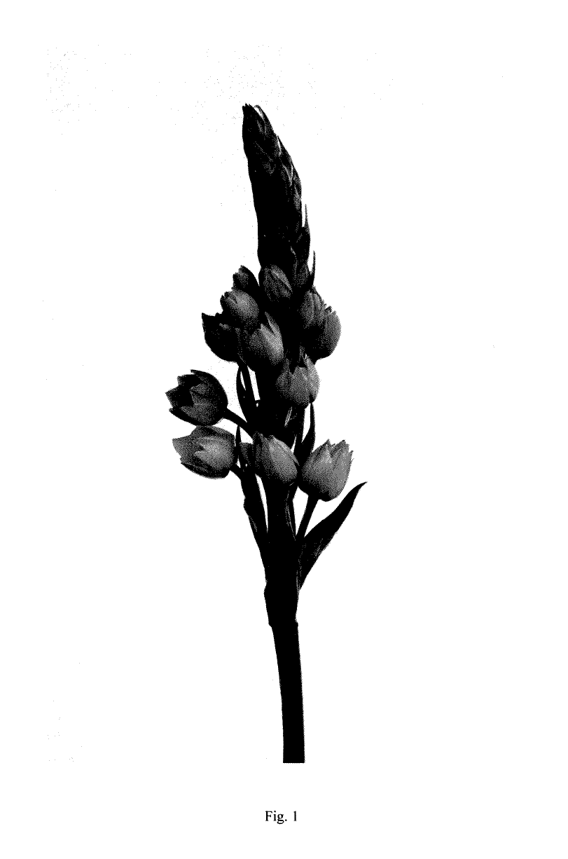

The accompanying photograph in FIG. 1 illustrates in full color a typical flowering stem of ‘MFPRINSTAR’ grown in a nursery. The photograph was taken using conventional techniques and although colors may appear different from actual colors due to light reflectance it is as accurate as possible by conventional photographic techniques.

### DETAILED BOTANICAL DESCRIPTION

In the following description, color references are made to the Royal Horticultural Society Mini Colour Chart 2001 except where general terms of ordinary dictionary significance are used. The following observations and measurements describe ‘MFPRINSTAR’ plants grown outdoors and in greenhouse during the Spring, and Summer in Moshav Givat Chen, Israel. The growing temperature ranged from 20° C. to 35° C. during the day and from 17° C. to 23° C. during the night. General light conditions are bright, normal sunlight. Measurements and numerical values represent averages of typical plant types.
Botanical classification: Ornithogalum ‘MFPRINSTAR’
Propagation:
   Type of propagation typically used.—Cutting.
   Time to initiate roots.—10-14 days.
   Root description.—Fibrous.
Plant:
   Age of plant described.—Approximately 5 months.
   Pot size of plant described.—Planted in soil.
   Typical time to produce a flowering plant commercially.—150-180 days.
   Height.—Approximately 40 cm to top of highest leaf. Approximately 60 cm to top of highest inflorescence.
   Plant spread.—Approximately 25 cm.
   Growth rate.—High.
   Branching characteristics.—Non-branching.
Foliage:
   Leaf.—Arrangement: Rosette Average Length: Approximately 25 cm. Average Width: Approximately 5 cm. Shape of blade: Sharp tip, flattened Apex: Sharp tip Base: Flattened Margin: Smooth Texture of top surface: Smooth Texture of bottom surface: Smooth Quantity of leaves per plant: Approximately 7 Venation: Type: Linear.
Flower:
   Natural flowering season.—Spring.
   Flowering stems produced per bulb.—2-3.
   Inflorescence type and habit.—Long raceme.
   Rate of flower opening.—3-5 days from pigments formation.
   Flower longevity on plant, or in vase if cut flower.—6 weeks and 3 weeks respectively.
   Quantity of flowers per inflorescence.—40.
   Average inflorescence per plant.—1.
   Inflorescence size.—Height: Approximately 30 cm. Width: Approximately 4 cm.
   Individual flowers.—Length: Approximately 3 cm Width: Approximately 3 cm Petals: Quantity: 6 Shape: Eliptic, concave Length: Approximately 1.5 cm Width: Approximately 1 cm Margin: Smooth Apex: Tip-like Base: Smooth Texture: Smooth Appearance: Matte.
   Color.—Petals: When opening: Outer surface: Near RHS NO25 Inner surface: Near RHS NO25 Fully opened: Outer surface: Near RHS NO25 Inner surface: Near RHS NO25 Color Changes when Aging: Yes. Become brown when petals getting old Sepals: Quantity: 40 Shape: Concave Blade-like Length: Approximately 2-5 cm Width: Approximately 1 cm Margin: Smooth Apex: Sharp tip Base: Smooth Texture: Smooth Appearance: Matte.
   Color.—Sepals: When opening: Green Outer surface: Near RHS Yellow-Green 144C Inner surface: Near RHS. Yellow-Green 144C Fully opened: Outer surface: Near RHS Yellow-Green 144C Inner surface: Near RHS Yellow-Green 144C.
   Bud.—Shape: Conic. Length: Approximately 1 cm. Diameter: Approximately 0.5 cm. Color: Green Near RHS.
   Pedicels.—Length: Approximately 3 cm. Width: Approximately 0.01 cm. Aspect: Angular Strength: Firm Texture: Smooth Color: Near RHS Yellow-Green 144A.
   Scape.—Length: Approximately 60 cm Width: Approximately 1 cm Texture: Smooth Angle: Erect Strength: High Color: Near RHS Green 137B.
   Fragrance.—None.
Reproductive organs:
   Stamens.—Number: 6 Filament Length: Approximately 0.4 cm. Filament Color: Near RHS Black 202A Anther length: 0.3 cm Anther color: Near RHS Yellow 3A Anther shape: Tag-like Pollen: Sphere with pits.
   Pistil.—Number: 1 Length: Approximately 0.3 cm. Style color: Near RHS Black 202A Stigma: Shape: Triple Color: Near RHS Black 202A.
Other characteristics:
   Seeds and fruits.—Completely fertile.
   Disease/pest resistance.—Resistance to Erwinia corotovora. Neither resistance nor susceptibility to other normal diseases and pests of Ornithogalum has been observed.
   Temperature tolerance.—Tolerates temperatures from approximately 1° C. to 30° C.

What is claimed is:

1. A new and distinct cultivar of Ornithogalum plant named ‘MFPRINSTAR’ as herein illustrated and described.
